(19) United States
(12) Patent Application Publication
(10) Pub. No.: US 2026/0265230 A1
(43) Pub. Date: Sep. 10, 2026

(54) FLUORESCENT AND SELF-ASSEMBLING UREIDOPYRIMIDINONE CONJUGATES AND PREPARATION THEREOF

(71) Applicant: COUNCIL OF SCIENTIFIC AND INDUSTRIAL RESEARCH AN INDIAN REGISTERED BODY INCORPOR, New Delhi (IN)

(72) Inventors: Rashid P P, Pune (IN); Gangadhar Jessy SANJAYAN, Pune (IN)

(73) Assignee: COUNCIL OF SCIENTIFIC AND INDUSTRIAL RESEARCH AN INDIAN REGISTERED BODY INCORPOR, New Delhi (IN)

(21) Appl. No.: 18/724,997

(22) PCT Filed: Feb. 7, 2023
(86) PCT No.: PCT/IN2023/050126

§ 371 Date: Jun. 27, 2024

(30) Foreign Application Priority Data
Feb. 7, 2022 (IN) 202211006558

(51) Int. Cl. C07D 405/04; C07D 239/47; C07K 19/00

(52) U.S. Cl. CPC C07D 405/04; C07D 239/47; C07K 19/00

(57) ABSTRACT

The present invention relates to fluorescent and self-assembling ureidopyrimidinones conjugates of formula (I). The present invention further relates to the process for preparation of compounds of Formula (I).

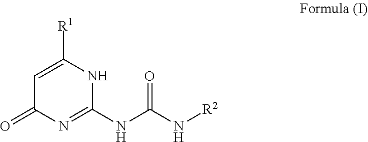

The coumarin and hydroquinone conjugates of formula (I) are useful for inducing peptide/protein assembly and recognition, and also to modulate protein/peptide secondary and tertiary structures

### FIELD OF THE INVENTION

The present invention relates to a fluorescent and self-assembling ureidopyrimidinones compound of formula (I).

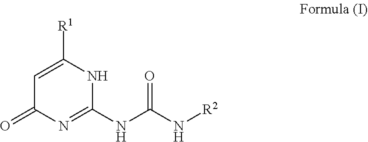

Particularly, the present invention relates to a process for the preparation of fluorescent and self-assembling ureidopyrimidinones compound of formula (I). More particularly, the present invention relates to ureidopyrimidinones compound of formula (I), useful for inducing peptide/protein assembly and recognition, and also to modulate protein/peptide secondary and tertiary structures.

### BACKGROUND OF THE INVENTION

The incorporation of fluorescent amino acids (FlAAs) into proteins and peptides is one of the most promising methods to investigate and understand various biophysical processes of biopolymers. FlAAs find extensive applications in studying protein-protein interactions, protein-peptide interactions and enzyme mechanisms by using handy fluorescent spectroscopy. Due to their low photostability and quantum yield, naturally occurring fluorescent amino acids such as tyrosine (Tyr), tryptophan (Trp), and phenylalanine (Phe) are hardly useful as an effective fluorophore for single-molecule measurements and imaging applications, especially under in vivo conditions. One of the alternative approaches involves design and application of unnatural fluorescent α-amino acids. FlAAs can be efficiently incorporated into protein structures with a high level of precision utilizing the conventional solid-phase method, unnatural amino acid mutagenesis, and expressed protein ligation (EPL). The photophysical properties of unnatural fluorescent α-amino acids can be tuned for a specific application. Several unnatural FlAAs have been made from various fluorophores shown below:

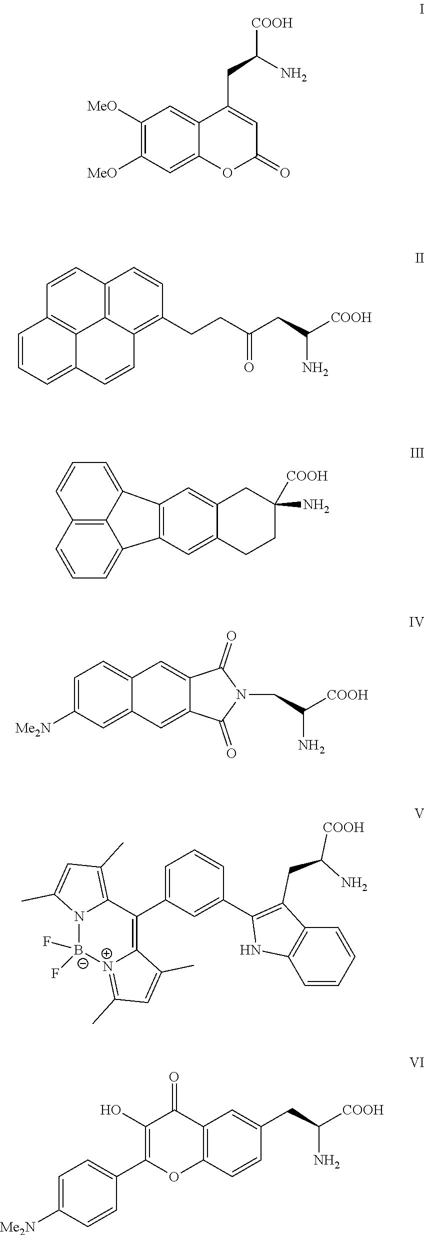

Among the various known fluorophores, the well-known coumarin motif shows high fluorescence quantum yield and large Stokes shifts, making it an excellent candidate to substitute green fluorescent protein (GFP) in the application of fluorescent labeling of living cells.

References may be made to Journal “Heteroatom Chemistry Volume 26, Number 5, 2015” which reports synthesis of an amino acid amide containing two benzamidine cores, one placed in the amide part the other one within the sulfonyl N-capping group. The amino acid side chain bears the 6,7-dimethoxycoumarin fluorophore. The fluorescent amino acid was prepared by using the von-Pechmann reaction. The two corresponding nitrile groups of a precursor molecule were simultaneously converted to the amidine moieties by the Pinner reaction.

Due to its high dimerization constant and easy synthetic accessibility, Meijer's ureidopyrimidinone (UPy) system has emerged as a promising building block for effecting peptide assembly. Bioactive supramolecular UPy-peptide conjugates obtained via a modular approach and their ability to self-assemble into fibrous structures have been elegantly described recently. Several bioactive peptide sequences were modified with a variety of UPy units in an effort to investigate their application scope. Convergent solid-phase synthesis strategies of UPy-modified peptides have been described recently. It is noteworthy that the modification of peptides with ureidopyrimidinone moieties offers considerable opportunities for biomedical applications and for developing biomaterials for tissue engineering.

Self-assembly in UPy is mediated by the strong AADD-type H-bonding with minimal repulsive secondary interactions. UPy self-assembly is characterized by its high dimerization strength (K_{dim})>1.9×107 M⁻¹ in CDCl₃) suitable for applications in diverse areas such as supramolecular polymers, biomaterial and catalysis as shown below:

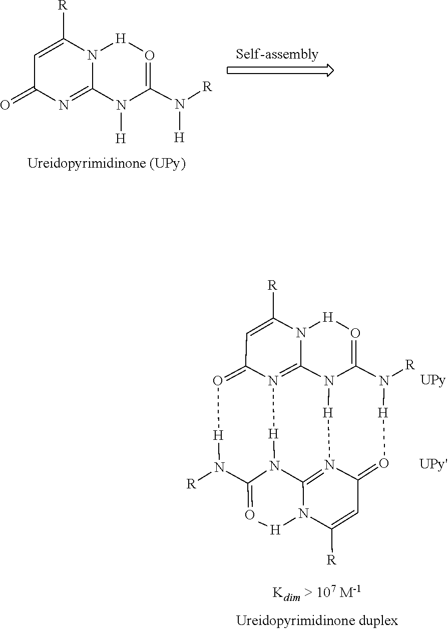

References may be made to Journal “Chem. Commun., 2012, 48, 1452-1454” which reports a synthesis of bioactive ureidopyrimidinone-based peptide conjugates, and their resultant self-assembly into fibrous structures. A convenient method to prepare supramolecular bio-conjugates in a facile and scalable manner using modular approach is self-assembling units and peptides are coupled using oxime chemistry.

References may be made to Journal “J. Org. Chem. 2017, 82, 12, 6403-6408”, which discloses a coumarin-conjugated self-assembling system adorned with valuable features such as high duplex stability and a built-in fluorophore, which would augment its application potential. However, the article fails to provide fluorescent amino acid-based compound which is capable of undergoing self-assembly.

In earlier reports, fluorescent amino acids are synthesized separately without having any self-assembling properties. Similarly there is no reported literature on the synthesis of self-assembling molecules labeled with fluorescent moiety.

There is an unmet need in the art to develop multi-purpose synthetic amino acids imparting both fluorescence and self-assembly properties so as to enhance the application scope.

### OBJECTIVE OF THE INVENTION

The main objective of the present invention is to provide a fluorescent and self-assembling ureidopyrimidinone compound of formula (I).

Another objective of the present invention is to provide a process of synthesis of the fluorescent and self-assembling ureidopyrimidinone compound of formula (I).

Yet another objective of the present invention is to provide the fluorescent and self-assembling ureidopyrimidinone conjugates blocks for inducing peptide/protein assembly and recognition and modulating protein/peptide secondary and tertiary structures which could be directly monitored by the handy tool fluorescence.

### ABBREVIATIONS USED

FlAAs—fluorescent amino acids
UPy—ureidopyrimidinone
Boc—tert-butyloxycarbonyl
CDI—carbonyldiimidazole
Bn—benzyl
Cbz—carboxybenzyl or benzyloxycarbonyl
HBTU—2-(1H-benzotriazol-1-yl)-1,1,3,3-tetramethyluronium hexafluorophosphate
HOBt—Hydroxybenzotriazole
BODIPY—boron-dipyrromethene
DMSO—Dimethyl sulfoxide
DIPEA—N,N-Diisopropylethylamine
TFA—Trifluoroacetic acid
DCM—dichloromethane
EPL—expressed protein ligation

### SUMMARY OF THE INVENTION

Accordingly, the present invention provides a fluorescent and self-assembling ureidopyrimidinone compound of formula (I)

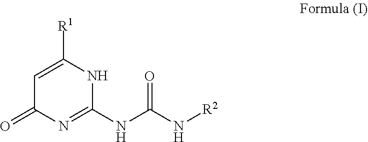

wherein:
R¹ represents

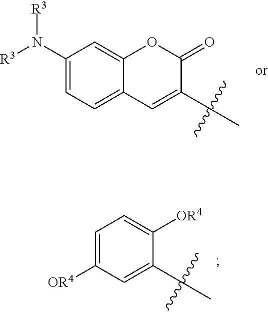

R² represents (un)substituted C₁-C₆ alkyl, (un)substituted aryl, (un)substituted natural amino acids which may be protected, (—CH₂)n-CH—(NH₂)(COOH), protected (—CH₂)n-CH—(NH₂)(COOH), linear or branched (un)substituted C₁-C₆ alcohols, or linear or branched (un)substituted C₁-C₆ amines; wherein n is 1-5; and
R³ and R⁴ independently represent (un)substituted C₁-C₆ alkyl, (un)substituted aryl, (un)substituted natural amino acids which may be protected, linear or branched (un)substituted C₁-C₆ alcohols, or linear or branched (un)substituted C₁-C₆ amines;
provided that at least one of R₂ or R₃ recite (un)substituted natural amino acids which may be protected, (—CH₂)n-CH—(NH₂)(COOH), protected (—CH₂)n-CH—(NH₂)(COOH), linear or branched (un)substituted C₁-C₆ alcohols, or linear or branched (un)substituted C₁-C₆ amines; wherein n is 1-5.

In an embodiment of the present invention, R¹ is 7-diethylamino-2-oxo-2H-chromen-3-yl or 1,4-dimethoxy phenyl.

In another embodiment of the present invention, R² is (un)substituted natural amino acids which may be protected and linear or branched (un)substituted C₁-C₆ amines.

In yet another embodiment of the present invention, the amino acid is selected from lysine, alanine, leucine, valine, phenylalanine, or combination thereof.

In yet another embodiment of the present invention, the compound of formula (I) is selected from the group consisting of:
i. N6-((4-(7-(diethylamino)-2-oxo-2H-chromen-3-yl)-6-oxo-1,6-dihydropyrimidin-2-yl) carbamoyl) lysine (1);
ii. N6-((6-(2,5-dimethoxyphenyl)-4-oxo-1,4-dihydropyrimidin-2-yl)carbamoyl)lysine (2);
iii. N²-(tert-butoxycarbonyl)-N⁶-((4-(7-(diethylamino)-2-oxo-2H-chromen-3-yl)-6-oxo-1,6-dihydropyrimidin-2-yl)carbamoyl)lysine (6);
iv. N²-(tert-butoxycarbonyl)-N6-((6-(2,5-dimethoxyphenyl)-4-oxo-1,4-dihydropyrimidin-2-yl)carbamoyl)lysine (10);
v. Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylleucinate (11);
vi. Methyl N6-((4-(7-(diethylamino)-2-oxo-2H-chromen-3-yl)-6-oxo-1,6-dihydropyrimidin-2-yl)carbamoyl)lysylleucinate (12);
vii. Methyl N²-((tert-butoxycarbonyl)alanyl)-N⁶-((4-(7-(diethylamino)-2-oxo-2H-chromen-3-yl)-6-oxo-1,6-dihydropyrimidin-2-yl)carbamoyl)lysylleucinate (13);
viii. Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylalanylleucinate (14);
ix. Methyl N6-((4-(7-(diethylamino)-2-oxo-2H-chromen-3-yl)-6-oxo-1,6-dihydropyrimidin-2-yl)carbamoyl)lysylalanylleucinate (15); and
x. Methyl N2-(tert-butoxycarbonyl)phenylalanylvalyl-N6-((4-(7-(diethylamino)-2-oxo-2H-chromen-3-yl)-6-oxo-1,6-dihydropyrimidin-2-yl)carbamoyl)lysylalanylleucinate (16).

In yet another embodiment, the present invention provides a process for synthesis of fluorescent and self-assembling ureidopyrimidinone compound of formula (I) comprising the steps of:
a) reacting compound of formula 4′ with carbonyldiimidazole (CDI) in a solvent to obtain the intermediate of formula 5′;

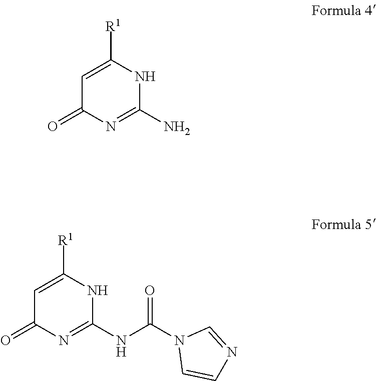

b) reacting the intermediate of formula 5′ as obtained in step (a) with Na-Boc-protected amino acid (Na-Boc-L-lysine) in presence of a base in the solvent to obtain BOC-protected compound of formula 6′;

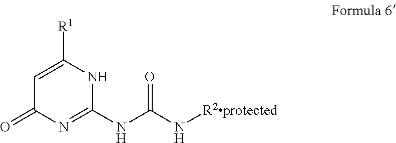

c) deprotecting the compound of formula 6′ as obtained in step (a) by using equal proportion of TFA and DCM to obtain the compound of formula (I);

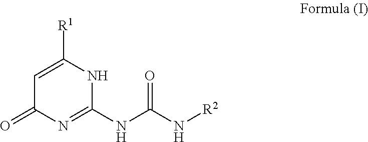

wherein: R¹ and R² are as defined herein.

In yet another embodiment of the present invention, the solvent is selected from polar or non-polar, or protic or aprotic solvents.

In yet another embodiment of the present invention, the solvent is selected from dimethyl sulfoxide, lower alcohols, nitriles, ketones, halogenated hydrocarbons, TFA, or combinations thereof.

In yet another embodiment of the present invention, the base is selected from ethylamine, triethylamine, DIPEA, pyridine, sodium hydroxide, alkali or alkaline earth metal carbonates, alkali or alkaline earth metal bicarbonates, or combinations thereof.

In yet another embodiment of the present invention, the process further comprises a step of incorporating the compound of formula (I) into peptides by reacting the compound of formula (I) with amino acids using coupling reagent, wherein the coupling reagent is hexafluorophosphate benzotriazoletetramethyluronium (HBTU).

In yet another embodiment of the present invention, the coumarin and hydroquinone conjugated with ureidopyrimidinones of formula (I) can be incorporated into proteins and peptides.

In yet another embodiment of the present invention, the coumarin and hydroquinone conjugated with ureidopyrimidinones are useful for inducing peptide/protein assembly and recognition.

In yet another embodiment of the present invention, the coumarin and hydroquinone conjugated with ureidopyrimidinones can be used to modulate the secondary and tertiary structures of peptides and proteins through strong quadruple H-bonding interactions.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

FIG. 1 represents absorption and emission spectra of (a) coumarin-UpyFlAA (6) and (b) hydroquinone-UPy-FlAA(10), in accordance with an implementation of the present disclosure.

FIG. 2 represents a process of preparing ureidopyrimidinone conjugate UPy-FlAA (1) wherein reagents and conditions are (i) 4-(diethylamino)salicylaldehyde, piperidine (cat.), EtOH, 24 h, reflux; (ii) CDI, DMSO, 80° C., 2 h; (iii) Na-Boc-L-lysine, DIPEA, DMSO, 60° C., 1 h; (iv) TFA:DCM (1:1), 2 h, RT, in accordance with an implementation of the present disclosure.

FIG. 3 represents a process of preparing ureidopyrimidinone conjugate UPy-FlAA (2) wherein reagents and conditions are (i) quanidine carbonate, EtOH, reflux, 24 h; (ii) CDI, DMSO, 80° C., 2 h; (iii) Na-Boc-L-lysine, DIPEA, DMSO, 60° C., 1 h; (iv) TFA:DCM (1:1), RT, 2 h, in accordance with an implementation of the present disclosure.

FIG. 4 represents a process for incorporation of compound UPy-FlAA 6 into peptides; wherein reaction conditions are (i) HCl·NH₂-Leu-OMe, HBTU, HOBt, DIPEA, DMF, RT, 6 h; (ii) TFA:DCM (1:1), RT, 2 h; (iii) Boc-Ala-OH, HBTU, HOBt, DIPEA, DMF, RT, 6 h; (iv) TFA·NH₂-Ala-Leu-OMe, HBTU, HOBt, DIPEA, DMF, RT, 6 h; (v) Boc-Phe-Val-OH, HBTU, HOBt, DIPEA, DMF, RT, 6 h, in accordance with an implementation of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The invention has other advantages and features which will be more readily apparent from the following detailed description of the invention, when taken in conjunction with the accompanying drawings:

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate parts throughout the view. Additionally, a reference to the singular includes a reference to the plural unless otherwise stated or inconsistent with the disclosure herein.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term, “(C₁₋₆) alkyl”, as used herein, refers to saturated aliphatic groups, including straight or branched-chain alkyl groups having six or fewer carbon atoms in its backbone, for instance, C₁₋₆ for straight chain and C₃-C₆ for branched chain. As used herein, (C₁₋₆) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The “aryl” as used herein refers to monocyclic or bicyclic hydrocarbon groups having 6 to 10 ring carbon atoms, wherein at least one carbocyclic ring is having a π electron system. Examples of aryl ring systems include, but are not limited to, phenyl and naphthyl. Unless indicated otherwise, aryl group can be unsubstituted or substituted with one or more substituents, for example 1-4 substituents independently selected from the group consisting of halogen, (C1-6) alkyl, hydroxy, cyano, nitro, —COOH and amino.

The “alcohols” as used herein refers to —OH or —O— group with alkyl, aryl, alkenyl, alkynyl, alkylaryl, arylalkyl heterocyclyl, aryl, heteroaryl, alkylheteroaryl, heteroarylalkyl, or other substituents, moiety or group as defined or disclosed herein, connected to the Markush.

The “amines” as used herein refers to —NH or —N— group with alkyl, aryl, alkenyl, alkynyl, alkylaryl, arylalkyl heterocyclyl, aryl, heteroaryl, alkylheteroaryl, heteroarylalkyl, or other substituent, moiety or group as defined or disclosed herein, connected to the Markush structure.

“Substituted” in the term “(un)substituted” used herein covers “optionally substituted alkyl”, “optionally substituted alkenyl”, “optionally substituted alkynyl”, “optionally substituted alkylaryl”, “optionally substituted arylalkyl”, “optionally substituted heterocyclyl”, “optionally substituted aryl”, “optionally substituted heteroaryl”, “optionally substituted alkylheteroaryl”, “optionally substituted heteroarylalkyl” and the like, mean an alkyl, alkenyl, alkynyl, alkylaryl, arylalkyl heterocyclyl, aryl, heteroaryl, alkylheteroaryl, heteroarylalkyl, or other substituents, moiety or group as defined or disclosed herein that has a substituent(s) that optionally replaces a hydrogen atom(s) or a substituent(s) that interrupts a carbon atom chain. Such substituents are as described herein. For a phenyl moiety, the arrangement of any two substituents present on the aromatic ring can be ortho (o), meta (m), or para (p). An optionally substituted fluoroalkyl is an alkyl or cycloalkyl moiety, typically a linear alkyl, wherein one or more hydrogen atoms is replaced by fluorine and at least one other atom other than carbon and fluorine. An optionally substituted or substituted substituent, moiety or group includes those having one or more additional group(s) that replace its hydrogen atom(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and di-substituted amino groups, and the protected derivatives thereof. By way of example and not limitation an optional substituent(s) may be halide, —CN, —NO₂, or LsRs, wherein each Ls is independently selected from a bond, —O—, —C(═O)—, —C(═O)O—, —S—, —S(═O)—, —S(═O)₂—, —NH—, —NHC(═O)—, C(═O)NH—, S(═O)₂NH—, —NHS(═O)₂, —OC(═O)NH—, —NHC(═O)O—, or —(C₁-C₆ alkylene)-; and each Rs is selected from H, alkyl, fluoroalkyl, heteroalkyl, cycloalkyl, aryl, heteroaryl, or heterocycloalkyl. The protecting groups that may form the protective derivatives of the above substituents may be found in sources such as Greene and Wuts, above. Optional substituents include those selected from the group consisting of halogen, —CN, —NH₂, —OH, —N(CH₃)₂, alkyl, fluoroalkyl, heteroalkyl, cycloalkyl, heterocycloalkyl, aryl, heteroaryl, alkoxy, aryloxy, alkylthio, arylthio, alkylsulfoxide, arylsulfoxide, alkylsulfone, and arylsulfone, those selected from the group consisting of halogen, —CN, —NH₂, —OH, NH(CH₃), —N(CH₃)₂, —CO₂H, —CO₂alkyl, —C(═O)NH₂, —C(═O)NHalkyl, —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, —S-alkyl and —S(═O)₂alkyl or those selected from the group consisting of halogen, —CN, —NH₂, —OH, —NH(CH₃), —N(CH₃)₂, —CH₃, —CH₂CH₃, —CF₃, —OCH₃, and —OCF₃. Typically, optionally substituted, substituent, moiety or group is substituted with one or two of the preceding groups, or more typically with one of the preceding groups. An optional substituent on an aliphatic carbon atom (acyclic or cyclic, saturated or unsaturated carbon atoms, excluding aromatic carbon atoms) further includes oxo (═O).

“Protecting group” as used here means a moiety that prevents or reduces the ability of the atom or functional group to which it is linked from participating in unwanted reactions. Non-limiting examples are for —OR^{PR}, wherein R^{PR} is a protecting group for the oxygen atom found in hydroxyl, while for —C(O)—OR^{PR}, R^{PR} may be carboxylic acid protecting group; for —SR^{PR}, R^{PR} may be protecting group for sulfur in thiols and for —NHR^{PR} or —N(R^{PR})₂—, at least one of R^{PR} is nitrogen atom protecting group for primary or secondary amines. Hydroxyl, amine, ketones, and other reactive groups may require protection against reactions taking place elsewhere in molecule. The protecting groups for oxygen, sulfur or nitrogen atoms are usually used to prevent unwanted reactions with electrophilic compounds, such as acylating agents. For example, the terms “amino-protecting group”, “oxygen-protecting group” and “nitrogen protecting group” are used interchangeably herein and refer to those organic groups intended to protect nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), -p-Methoxybenzyl (PMB), N-methylated amino acids and the like. Skilled persons will know how to choose group for ease of removal and for ability to withstand the disclosed process reactions.

The present invention provides a novel class of ureidopyrimidinones conjugates of formula (I). Coumarins and hydroquinones are efficient fluorophores and have proven application potential in diverse areas ranging from material to biomolecular sciences. On the other hand, UPy offers considerable application scope in biomaterial sciences owing to its strong self-assembling property involving the AADD-type quadruple hydrogen bonding interactions.

The present invention relates to fluorescent and self-assembling ureidopyrimidinone compound of formula (I)

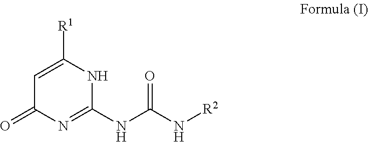

wherein:
R¹ represents

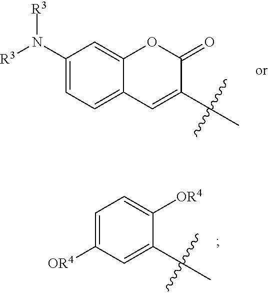

R² represents (un)substituted C₁-C₆ alkyl, (un)substituted aryl, (un)substituted natural amino acids which may be protected, (—CH₂)n-CH—(NH₂)(COOH), protected (—CH₂)n-CH—(NH₂)(COOH), linear or branched (un)substituted C₁-C₆ alcohols, or linear or branched (un)substituted C₁-C₆ amines; wherein n is 1-5; and R³ and R⁴ independently represent (un)substituted C₁-C₆ alkyl, (un)substituted aryl, (un)substituted natural amino acids which may be protected, linear or branched (un)substituted C₁-C₆ alcohols, or linear or branched (un)substituted C₁-C₆ amines; provided that at least one of R² or R³ recite (—CH₂)n-CH—(NH₂)(COOH), protected (—CH₂)n-CH—(NH₂)(COOH), (un)substituted natural amino acids which may be protected, linear or branched (un)substituted C₁-C₆ alcohols, or linear or branched (un)substituted C₁-C₆ amines.

The amino acid is selected from lysine, alanine, leucine, valine, phenylalanine, or combinations thereof.

The present invention provides a process for synthesis of fluorescent and self-assembling ureidopyrimidinone conjugates of formula (I) comprising the steps of:
a) reacting compound of formula 4′ with carbonyldiimidazole (CDI) in a solvent to obtain the intermediate of formula 5′;

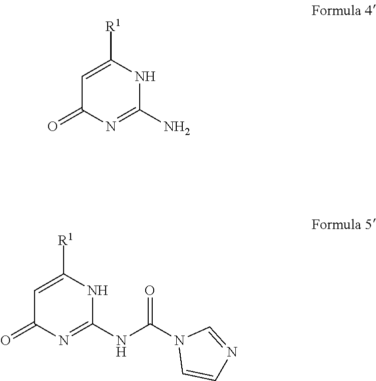

b) reacting the intermediate of formula 5′ as obtained in step (a) with Na-Boc-protected amino acid (Na-Boc-L-lysine) in presence of a base in a solvent to obtain BOC-protected compound of formula 6′;

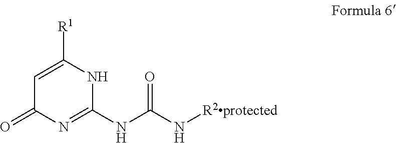

c) deprotecting the compound of formula 6′ as obtained in step (a) by using equal proportion of TFA and DCM to obtain the compound of formula (I);

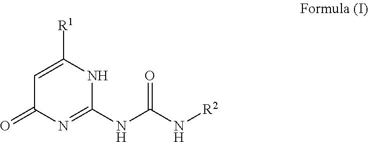

wherein: R¹ and R² are as defined earlier.

The process of preparing ureidopyrimidinone conjugate of formula UPy-FlAA (1) is depicted in FIG. 2. According to the process, compound (3) is reacted with 4-(diethylamino)salicylaldehyde in presence of a catalyst and a solvent to obtain compound (4), which is further reacted with CDI in presence of the solvent to yield an intermediate compound (5). The intermediate compound (4a) is reacted with Boc-protected amino acid (Nα-Boc-L-lysine) in presence of the solvent and a base to yield the Boc-protected coumarin-UpyFlAA (6) which is Boc-deprotected in TFA-DCM (1:1) to obtain compound UPy-FlAA (1). The catalyst is selected from organic amine bases such as piperidine.

In the present process, the solvent for the reaction is selected from polar or nonpolar, protic or aprotic solvent such as lower alcohols, nitriles, ketones, halogenated hydrocarbons, TFA, or combinations thereof.

The base for the reaction is selected from organic base such as ethylamine, triethylamine, DIPEA, pyridine or from inorganic base such as sodium hydroxide, alkali or alkaline earth metal carbonates, alkali or alkaline earth metal bicarbonates, or combinations thereof.

The present invention discloses a process for synthesis of fluorescent and self-assembling ureidopyrimidinone conjugates of formula UPy-FlAA (2) as shown in FIG. 3. According to the process, compound (7) is reacted with guanidine carbonate in solvent to obtain compound (8) which is further reacted with CDI in presence of solvent to yield an intermediate compound (9). The intermediate (9) is reacted with Boc-protected amino acid in presence of solvent and base to yield the Boc-protected hydroquinone-UPyFlAA (10) which is Boc-deprotected in TFA-DCM (1:1) to obtain ureidopyrimidinone conjugates of formula UPy-FlAA (2).

The solvent for the process is selected from polar or nonpolar, protic or aprotic solvent such as lower alcohols, nitriles, ketones, halogenated hydrocarbons, TFA, or combinations thereof.

The base for the reaction is selected from organic base such as ethylamine, triethylamine, DIPEA, pyridine or from inorganic base such as sodium hydroxide, alkali or alkaline earth metal carbonates, alkali or alkaline earth metal bicarbonates, or combinations thereof.

The ureidopyrimidinones conjugates are incorporated into proteins and peptides.

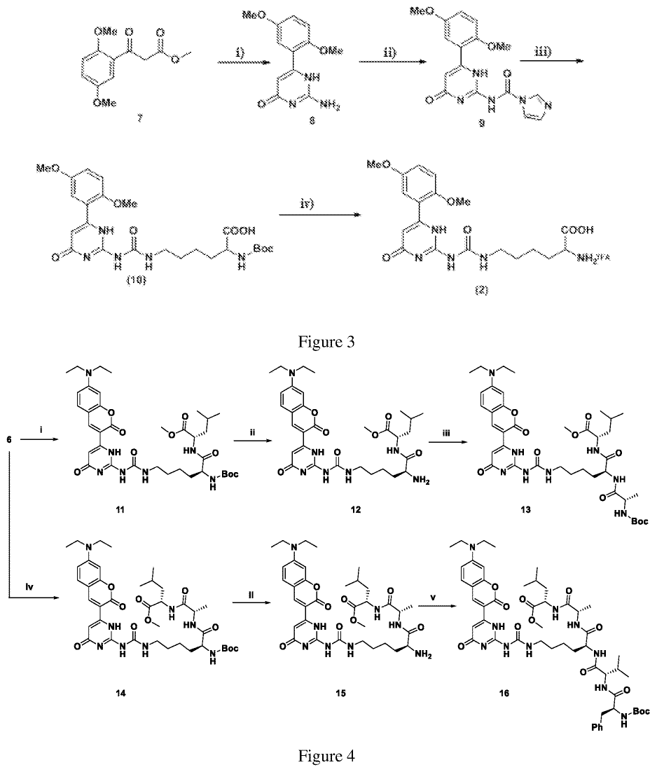

The present invention relates to a process for incorporation of couamrin-UPyFlAA (6) into peptides, comprising the steps as shown in FIG. 4, wherein reaction conditions: (i) HCl·NH₂-Leu-OMe, HBTU, HOBt, DIPEA, DMF, RT (room temperature), 6 h; (ii) TFA:DCM (1:1), RT, 2 h; (iii) Boc-Ala-OH, HBTU, HOBt, DIPEA, DMF, RT, 6 h; (iv) TFA·NH₂-Ala-Leu-OMe, HBTU, HOBt, DIPEA, DMF, RT, 6 h; (v) Boc-Phe-Val-OH, HBTU, HOBt, DIPEA, DMF, rt, 6 h.

Due to the weak solubility and reactivity of the couamrin-UPyFlAA amino acids, the approach or process as explained in FIG. 4 is extremely viable. According to the process, compound (6) is coupled at C-terminus with NH₂-Leu-OMe in presence of coupling reagent and a base to obtain compound (11) which is further deprotected using 1:1 TFA-DCM to afford the free amine (12). Further coupling of compound (12) at N-terminus with Boc-Ala-OH using a coupling agent afford the tripeptide (13).

The compound (6) is coupled at C-terminus with dipeptide NH₂-Ala-Leu-OMe in presence of coupling reagent and a base to obtain compound (14) which is further deprotected using 1:1 TFA-DCM to afford free amine (15). Further coupling of compound (15) at N-terminus with Boc-Phe-Val-OH using coupling reagent and a base provides the pentapeptide (16).

The solvent for the process is selected from polar or nonpolar, protic or aprotic solvent such as lower alcohols, nitriles, ketones, halogenated hydrocarbons, TFA, or combinations thereof. The base for the reaction is selected from organic base such as ethylamine, triethylamine, DIPEA, pyridine or from inorganic base such as sodium hydroxide, alkali or alkaline earth metal carbonates, alkali or alkaline earth metal bicarbonates or combinations thereof.

The coupling reagent for reaction is selected from standard coupling reagents commonly used for the activation of free carboxylic acids during the solution and solid phase peptide synthesis such as HBTU.

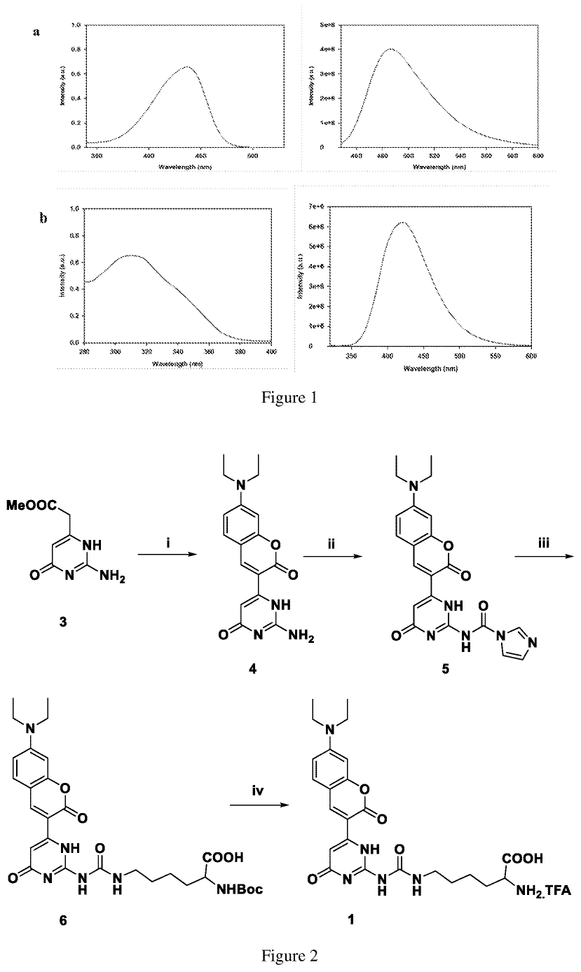

Fluorescent studies of compounds Boc-protected coumarin UPyFlAA 6 and hydroquinone-UPyFlAA10 were performed in DMSO at the concentration of 1×10⁻⁵ M. Coumarin and hydroquinone-UPyFlAAs 6 and 10, respectively, showed high level of fluorescence and the relative fluorescence intensity was measured at various excitation wavelengths to determine their maximum absorption wavelength (λabs) and maximum emission wavelength (λems). Coumarin-UPyFlAA6 showed maximum λabs at 437 nm with a maximum λems at 487 nm, while the hydroquinone-UPyFlAAs10 showed maximum λabs at 310 nm with a maximum λems at 421 nm (FIG. 1).

### EXAMPLES

Following examples are given by way of illustration and therefore should not be construed to limit the scope of the invention.

### Experimental

Unless otherwise stated, all reagents were purchased at the highest commercial quality and used without further purification. Compound 3 and 4 were synthesized as per the reported procedure. The compound 8 was readily synthesized in three steps, following the literature procedures. Boc-protected amino acids and dipeptides were synthesized as per the reported procedures. All reactions were carried out with laboratory grade solvent and purity of intermediates and final compounds were checked on precoated silica gel G254 TLC plates (Merck). Visualization of spot on TLC was achieved by the use of UV light (254 nm) and ninhydrin test. All synthesized compounds were purified on silica gel column chromatographic by using 100-200, 230-400 Mesh Silica gel.

### Synthesis of the compounds of Formula (I)

### Example 1: Synthesis of UPy-FlAA (1)

### Step [1]: Synthesis of N-(6-(7-(diethylamino)-2-oxo-2H-chromen-3-yl)-4-oxo-1,4-dihydropyrimidin-2-yl)-1H-imidazole-1-carboxamide (5)

A solution containing compound 4 (3 g, 9.1 mmol, 1 equiv) and CDI (2.97 g, 18.3 mmol, 2 equiv) in 20 mL of dry DMSO was heated at 80° C. under N2 atmosphere for 2 h. The reaction mixture was cooled to room temperature [25° C.] and diluted with acetone. The resulting precipitate was filtered, washed thoroughly with acetone, and dried under vacuum to give the CDI intermediate 5 as an orange solid, which was used for the next reaction without further purification. Yield: 2.7 g, 72%

### Step [2]: Synthesis of N2-(tert-butoxycarbonyl)-N6-((4-(7-(diethylamino)-2-oxo-2H-chromen-3-yl)-6-oxo-1,6-dihydropyrimidin-2-yl)carbamoyl)lysine UPyFlAA (6)

To a solution of CDI intermediate 5 (2 g, 4.7 mmol, 1 equiv), 20 mL of dry DMSO was added, with Na-Boc-L-lysine (1.76 g, 7.14 mmol, 1.5 equiv) and DIPEA (2.94 mL, 14.2 mmol, 3 equiv) and the reaction mixture was heated at 60° C. under N2 atmosphere for 2 h. After completion of the reaction, the reaction mixture was cooled to room temperature (27° C.) and poured into water, and the aqueous layer was washed with ethyl acetate two times and acidified with aqueous KHSO₄. The resulting precipitate was filtered, washed with diethyl ether and dried under vacuum to give desired product 6 as a yellow colored solid, which was carried forward for the next reaction.

Yield: 2.3 g, 65%, [α]27D: −167.88° (c=0.3, DMSO); mp>300° C.; ¹H NMR (500 MHz, DMSO-d6) δ: 11.76 (br. s., 1H), 9.78 (br. s., 1H), 8.56 (s, 1H), 7.46 (d, J=9.0 Hz, 1H), 7.27 (br. s., 1H), 7.06 (d, J=8.1 Hz, 1H), 6.94 (s, 1H), 6.72 (dd, J=2.5, 9.0 Hz, 1H), 6.52 (d, J=2.5 Hz, 1H), 3.91-3.81 (m, 1H), 3.47-3.42 (m, 4H), 3.18-3.11 (m, 2H), 1.73-1.44 (m, 4H), 1.43-1.30 (m, 11H), 1.17-1.08 (m, 6H); ¹³C NMR (125 MHz, DMSO-d6) δ: 174.30, 159.37, 156.67, 155.67, 154.79, 151.90, 143.98, 130.91, 112.90, 109.80, 107.73, 95.81, 78.00, 53.45, 44.34, 39.51, 38.97, 30.49, 28.83, 28.23, 23.04, 163.4, 12.37; HRMS (ESI) calcd [M+H]⁺ for C29H39N6O8599.2824, found 599.2822.

### Step [3]: Synthesis of N6-((4-(7-(diethylamino)-2-oxo-2H-chromen-3-yl)-6-oxo-1,6-dihydropyrimidin-2-yl) carbamoyl) lysine UPy-FlAA (1)

Compound 6 0.5 g (1.6 mmol, 1 equiv) was dissolved in 5 ml of DCM followed by addition of 5 ml of TFA at 0-5° C. and the reaction mixture was stirred for 2 h at room temperature (30° C.). After the completion of the reaction, the solvent was stripped off and co-evaporated using toluene to obtain the desired compound 1 as a yellow colored solid;

Yield: 0.41 g, 96%, [α]²⁷D: −164.24° (c=0.2, DMSO); mp: 198° C.; 1H NMR (500 MHz, DMSO-d6) δ: 11.71 (br. s., 1H), 10.18-9.84 (m, 1H), 8.59 (s, 1H), 8.36-8.17 (m, 3H), 7.98-7.77 (m, 1H), 7.51-7.37 (m, 2H), 6.94 (s, 1H), 6.78- 6.69 (m, 1H), 6.58-6.46 (m, 1H), 3.98-3.85 (m, 1H), 3.52-3.39 (m, 4H), 3.23-3.08 (m, 2H), 1.87-1.72 (m, 2H), 1.56-1.31 (m, 4H), 1.13 (t, J=7.0 Hz, 6H); 13C NMR (125 MHz, DMSO-d6) δ: 171.11, 159.38, 156.68, 154.94, 144.08, 130.86, 109.84, 107.75, 103.87, 95.83, 51.93, 44.33, 38.74, 29.69, 28.77, 27.11, 21.67, 12.35; HRMS (ESI) calcd [M+H]+ for C24H31N6O6 499.2300, found 499.2297.

### Example 2: Synthesis of UPy-FlAA (2)

### Step [1]: Synthesis of N-(4-(2,5-dimethoxyphenyl)-6-oxo-1,6-dihydropyrimidin-2-yl)-1H-imidazole-1-carboxamide (9)

A solution containing compound 8 (3 g, 12.13 mmol, 1 equiv) and CDI (3.93 g, 24.27 mmol, 2 equiv) in 20 mL of dry DMSO was heated at 80° C. under N2 atmosphere for 2 h. The reaction mixture was cooled to 25° C. room temperature and diluted with acetone. The resulting precipitate was filtered, washed thoroughly with acetone, and dried under vacuum to give the CDI intermediate 9 as a colorless solid, which was used for the next reaction without further purification. Yield: 3.4 g, 82%,

### Step [2]: Synthesis of N2-(tert-butoxycarbonyl)-N6-((6-(2,5-dimethoxyphenyl)-4-oxo-1,4-dihydropyrimidin-2-yl)carbamoyl)lysine (10) or UPyFlAA (10)

A solution of CDI intermediate 9 (2 g, 5.86 mmol, 1 equiv), Na-Boc-L-lysine (2.16 g, 8.79 mmol, 1.5 equiv) and DIPEA (3.25 mL, 17.58 mmol, 3 equiv) in 20 mL of dry DMSO was heated at 60° C. under N₂ atmosphere for 1 h. After completion of reaction, the reaction mixture was cooled to 27° C. room temperature and poured into water. The aqueous layer washed with ethyl acetate and then acidified with aqueous KHSO₄, The resulting precipitate was filtered, washed with diethyl ether and dried under vacuum to give desired product 10 as a colorless solid;

Yield: 2.3 g, 75%, [α]²⁷D: −169.24° (c=0.1, DMSO); mp>300° C.; 1H NMR (400 MHz, DMSO-d6) δ: 11.90 (br. s., 1H) 9.78 (br. s., 1H) 7.47 (br. s., 1H) 7.33 (br. s., 1H) 6.96-7.11 (m, 3H) 6.67 (d, J=6.63 Hz, 0H) 6.48 (br. s., 1H) 3.82-3.88 (m, 1H) 3.80 (s, 3H) 3.74 (s, 3H) 3.14 (q, J=5.96 Hz, 2H) 1.53-1.71 (m, 2H) 1.41-1.49 (m, 2H) 1.31-1.39 (m, 11H); 13C NMR (100 MHz, DMSO-d 6) δ: 174.25, 159.40, 155.61, 154.69, 153.00, 151.65, 151.30, 126.07, 116.33, 114.99, 113.54, 106.71, 77.96, 56.16, 55.53, 53.41, 39.51, 30.47, 28.83, 28.20, 27.96, 22.98; HRMS (ESI) calcd [M+H]+ for C24H33N5O8 520.2402, found 520.2397.

### Step [3]: Synthesis of N6-((6-(2,5-dimethoxyphenyl)-4-oxo-1,4-dihydropyrimidin-2-yl)carbamoyl)lysine, UPy-FlAA (2)

0.5 g (0.96 mmol, 1 equiv) of compound 10 was dissolved in 5 ml of DCM followed by addition of 5 ml of TFA at 0-5° C. and stirred for 2 h at 30° C. room temperature. After the completion of reaction, the solvent was stripped off and co-evaporated using toluene to obtain the desired compound 2 as a colorless solid;

Yield: 0.48 g, 98%, [α]²⁷D: −152.88° (c=0.1, DMSO); mp: 210° C.; 1H NMR (400 MHz, DMSO-d6) δ: 11.83 (br. s., 1H) 9.99 (br. s., 0H) 8.29 (br. s., 3H) 7.76 (br. s., 1H) 7.36 (br. s., 1H) 6.97-7.11 (m, 2H) 6.50 (s, 1H) 3.88 (br. s., 1H) 3.80 (s, 3H) 3.74 (s, 3H) 3.16 (d, J=5.50 Hz, 2H) 1.70-1.88 (m, 2H) 1.27-1.55 (m, 4H); ¹³C NMR (100 MHz, DMSO-d6) δ: 171.14, 158.89, 158.56, 155.01, 153.09, 151.75, 116.69, 114.98, 113.64, 106.54, 56.22, 55.56, 51.98, 40.42, 38.80, 29.72, 28.82, 21.72; HRMS (ESI) calcd [M+H]+ for C19H25N5O6 420.1878, found 420.1870.

### Incorporation of Couamrin-UPyFlAA (6) into Peptides

### Example 7: Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylleucinate (11)

To a solution of 6 (0.5 g, 0.83 mmol, 1 equiv) in dry DMF, DIPEA (0.58 mL, 3.3 mmol, 4 equiv) was added, followed by the addition of HBTU (0.47 g, 1.25 mmol, 1.5 equiv) and a catalytic amount of HOBt (0.05 g, mmol, 0.5 equiv) at 0-5° C. Then, HCl·NH₂-Leu-OMe (0.3 g, 1.67 mmol, 2 equiv) was added. The reaction mixture was then stirred at 30° C. for 6 h. The reaction mixture was poured into water and the resulting precipitate was filtered. The precipitate was dissolved in DCM and washed sequentially with saturated solutions of NaHCO₃, KHSO₄, water and brine. The organic layer was then dried with anhydrous Na₂SO₄ and evaporated under reduced pressure to obtain the crude product which was purified by column chromatography (eluent: DCM/MeOH (97:3), Rf: 0.6) to afford 11 as an orange solid.

Yield: 0.55 g, 89%; [α]²⁷D: −151.16° (c=0.1, DMSO); mp: 200-202° C.; ¹H NMR (500 MHz, DMSO-d6) δ: 11.65 (d, J=0.8 Hz, 1H), 9.74 (br. s., 1H), 8.56 (s, 1H), 8.12 (d, J=8.0 Hz, 1H), 7.47 (d, J=9.1 Hz, 1H), 7.17 (br. s., 1H), 6.95 (br. s., 1H), 6.84-6.66 (m, 2H), 6.53 (dd, J=0.4, 2.1 Hz, 1H), 4.35-4.25 (m, 1H), 3.99-3.86 (m, 1H), 3.64-3.55 (m, 3H), 3.51-3.39 (m, 4H), 3.20-3.06 (m, 2H), 1.69-1.47 (m, 6H), 1.39-1.28 (m, 10H), 1.13 (t, J=7.1 Hz, 6H), 0.92-0.79 (m, 8H); ¹³C NMR (125 MHz, DMSO-d6) δ: 172.90, 172.33, 159.31, 156.64, 155.26, 154.72, 151.88, 143.92, 130.87, 112.91, 109.77, 107.71, 104.24, 95.80, 77.97, 53.96, 51.78, 50.06, 44.30, 39.72, 39.09, 31.53, 28.93, 28.13, 24.07, 22.80, 22.71, 21.19, 12.33; HRMS (ESI) calcd [M+H]+ for C36H52N7O9 726.3821, found 726.3820.

### Example 8: Methyl N6-((4-(7-(diethylamino)-2-oxo-2H-chromen-3-yl)-6-oxo-1,6-dihydropyrimidin-2-yl)carbamoyl)lysylleucinate (12)

0.5 g of compound 11 was dissolved in 5 ml of DCM followed by addition of 5 ml of TFA at 0-5° C. and stirred for 1 h at 25° C. room temperature. After the completion of reaction, the solvent was stripped off and co-evaporated using toluene to obtain the TFA salt which was neutralized by saturated NaHCO₃, and the resulting precipitate was filtered, washed thoroughly with diethyl ether, and dried under vacuum to give the desired compound 12 as orange solid.

Yield: 0.4 g, 97%, [α]²⁷D: −168.32° (c=0.2, DMSO); mp>300° C.; ¹H NMR (400 MHz, DMSO-d6) δ: 8.89 (br. s., 1H), 8.44 (d, J=0.2 Hz, 1H), 8.35-8.16 (m, 1H), 7.44 (dd, J=0.5, 8.9 Hz, 1H), 6.89-6.77 (m, 1H), 6.75-6.62 (m, 1H), 6.51 (d, J=1.0 Hz, 1H), 4.35-4.25 (m, 1H), 3.81-3.70 (m, 1H), 3.60 (br. s., 3H), 3.49-3.39 (m, 4H), 3.18 (dd, J=5.4, 6.1 Hz, 2H), 1.63-1.43 (m, 7H), 1.19-1.06 (m, 6H), 0.94-0.72 (m, 8H); 13C NMR (100 MHz, DMSO-d6) δ: 174.59, 172.92, 159.37, 156.45, 154.95, 151.56, 143.04, 130.54, 114.07, 109.58, 107.79, 103.99, 95.81, 53.97, 51.79, 50.04, 44.26, 39.71, 39.09, 34.37, 29.25, 24.22, 22.74, 21.28, 12.34; HRMS (ESI) calcd [M+H]+ for C31H44N7O7626.3297, found 626.3296.

### Example 9: Methyl N2-((tert-butoxycarbonyl)alanyl)-N6-((4-(7-(diethylamino)-2-oxo-2H-chromen-3-yl)-6-oxo-1,6-dihydropyrimidin-2-yl)carbamoyl)lysylleucinate (13)

To a solution of Boc-Ala-OH (0.18 g, 0.95 mmol, 1.2 equiv) in dry DMF, DIPEA (0.3 mL, 2.4 mmol, 3 equiv) was added followed by the addition of HBTU (0.45 g, 1.2 mmol, 1.5 equiv), and a catalytic amount of HOBt (0.05 g, 0.3 mmol, 0.5 equiv) at 0-5° C. Finally, compound 12 (0.5 g, 0.79 mmol, 1 equiv) was added. The reaction mixture was stirred at 0° C. for 10 minutes and at 30° C. for 6 h. The reaction mixture was poured into water and the resulting precipitate was filtered. The precipitate was dissolved in DCM and washed sequentially with saturated solutions of NaHCO₃, KHSO₄, water and brine. The organic layer was then dried with anhydrous Na₂SO₄ and evaporated under reduced pressure to obtain the crude product which was purified by column chromatography (eluent: DCM/MeOH (95:5), Rf: 0.6) to afford 13 as a yellow solid (0.59 g, 92%).

Yield: 0.59 g, 92%, [α]²⁷D: −176.56° (c=0.1, DMSO); mp: 208-210° C.; ¹H NMR (500 MHz, DMSO-d6) δ: 11.67 (br. s., 1H) 9.75 (br. s., 1H) 8.58 (br. s., 1H) 8.02-8.36 (m, 1H) 7.70 (br. s., 1H) 7.49 (br. s., 1H) 7.16 (br. s., 1H) 6.95 (br. s., 1H) 6.75 (br. s., 1H) 6.54 (br. s., 1H) 4.28 (br. s., 2H) 3.95 (br. s., 1H) 3.60 (br. s., 3H) 3.46 (br. s., 4H) 3.14 (br. s., 2H) 1.42-1.73 (m, 7H) 1.36 (br. s., 10H) 1.14 (br. s., 9H) 0.78-0.97 (m, 6H); 13C NMR (125 MHz, DMSO-d6) δ: 172.75, 172.49, 171.64, 159.33, 156.66, 154.73, 151.90, 143.97, 130.90, 112.94, 109.78, 107.71, 104.32, 95.82, 78.10, 51.79, 50.17, 49.81, 44.31, 39.72, 39.31, 32.03, 28.98, 28.15, 24.17, 22.74, 22.24, 21.20, 17.69, 12.34; HRMS (ESI) calcd [M+H]+ for C39H57N8O10 797.4198, found 797.4198.

### Example 10: Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylalanylleucinate (14)

To a solution of compound 6 (0.5 g, 0.8 mmol, 1 equiv) in dry DMF, DIPEA (0.58 mL, 3.3 mmol, 4 equiv) was added, followed by the addition of HBTU (0.47 g, 1.2 mmol, 1.5 equiv), and a catalytic amount of HOBt (0.05 g, 0.4 mmol, 0.5 equiv) at 0-5° C. Then, HCl·NH₂-Ala-Leu-OMe (0.39 g, 1.25 mmol, 1.5 equiv) was added. The reaction mixture was stirred at 0-5° C. for 10 minutes and at 30° C. for 6 h. The reaction mixture was poured into water and the resulting precipitate was filtered. The precipitate was dissolved in DCM and washed sequentially with saturated solutions of NaHCO₃, KHSO₄, water and brine. The organic layer was then dried with anhydrous Na₂SO₄ and evaporated under reduced pressure to obtain the crude product which was purified by column chromatography (eluent: DCM/MeOH (95:5), Rf: 0.6) to afford 14 as a yellow solid.

Yield: 0.54 g, 81%, [α]²⁷D: −168° (c=0.1, DMSO); mp: 205-207° C.; ¹H NMR (400 MHz, DMSO-d6) δ: 11.41 (br. s., 1H), 9.85 (br. s., 1H), 8.57 (d, J=2.8 Hz, 1H), 8.28-8.17 (m, 1H), 7.87-7.78 (m, 1H), 7.48 (dd, J=2.4, 9.1 Hz, 1H), 6.99-6.86 (m, 2H), 6.74 (td, J=1.0, 9.3 Hz, 1H), 6.53 (d, J=2.4 Hz, 1H), 4.35-4.24 (m, 2H), 3.94-3.84 (m, 1H), 3.65-3.56 (m, 3H), 3.51-3.39 (m, 4H), 3.19-3.09 (m, 2H), 1.63-1.46 (m, 6H), 1.40-1.31 (m, 10H), 1.22-1.10 (m, 9H), 0.92-0.76 (m, 8H); ¹³C NMR (100 MHz, DMSO-d6) δ: 172.80, 172.32, 171.71, 159.33, 156.64, 155.37, 154.73, 151.87, 143.90, 130.86, 112.97, 109.77, 107.72, 104.08, 95.81, 78.03, 54.18, 51.83, 50.16, 47.65, 44.29, 39.71, 39.01, 31.53, 28.92, 28.14, 24.15, 22.84, 22.74, 21.21, 18.29, 12.33; HRMS (ESI) calcd [M+H]+ for C39H57N8O10 797.4194, found 797.4194.

### Example 11: Methyl N6-((4-(7-(diethylamino)-2-oxo-2H-chromen-3-yl)-6-oxo-1,6-dihydropyrimidin-2-yl)carbamoyl)lysylalanylleucinate (15)

0.5 g of compound 14 was dissolved in 5 ml of DCM followed by the addition of 5 ml of TFA at 0-5° C. and stirred for 1 h at 30° C. room temperature.

After the completion of reaction, the solvent was stripped off and co-evaporated using toluene to obtain the TFA salt which was neutralized by saturated NaHCO₃, and the resulting precipitate was filtered, washed thoroughly with diethyl ether, and dried under vacuum to give the desired compound 15 as an orange solid;

Yield: 0.42 g, 95%, [α]²⁷D: −138.56° (c=0.3, DMSO); mp: 207-209° C.; ¹H NMR (400 MHz, DMSO-d6) δ: 9.14 (br. s., 1H), 8.43 (d, J=2.13 Hz, 1H), 8.23-8.34 (m, 1H), 8.10-8.19 (m, 1H), 7.40-7.48 (m, 1H), 6.78 (br. s., 1H), 6.66-6.73 (m, 1H), 6.52 (br. s., 1H), 4.18-4.40 (m, 3H), 3.60 (br. s., 3H), 3.40-3.47 (m, 4H), 3.16-3.21 (m, 2H), 1.39-1.59 (m, 7H), 1.09-1.22 (m, 9H), 0.73-0.94 (m, 8H); 13C NMR (100 MHz, DMSO-d6) δ: 174.27, 172.83, 172.41, 159.40, 156.40, 155.00, 151.48, 142.83, 130.47, 114.41, 109.56, 107.82, 104.03, 95.84, 54.29, 51.85, 50.20, 47.46, 44.26, 39.72, 39.09, 34.40, 29.33, 24.20, 22.74, 21.24, 18.62, 12.35; HRMS (ESI) calcd [M+H]+ for C34H49N8O8 697.3668, found 697.3666.

### Example 12: Methyl N2-(tert-butoxycarbonyl)phenylalanylvalyl-N6-((4-(7-(diethylamino)-2-oxo-2H-chromen-3-yl)-6-oxo-1,6-dihydropyrimidin-2-yl)carbamoyl)lysylalanylleucinate (16)

To a solution of Boc-Phe-Val-OH (0.31 g, 0.86 mmol, 1.2 equiv) in dry DMF, DIPEA (0.37 mL, 2.1 mmol, 3 equiv) was added followed by the addition of HBTU (0.4 g, 1 mmol, 1.5 equiv) and a catalytic amount of HOBt (0.04 g, 0.3 mmol, 0.5 equiv) at 0-5° C. Then, compound 15 (0.5 g, 0.7 mmol, 1 equiv) was added. The reaction mixture was stirred at 0-5° C. for 10 minutes and at 30° C. for 6 h. The reaction mixture was poured into water and the resulting precipitate was filtered. The precipitate was dissolved in DCM and washed sequentially with saturated solutions of NaHCO3, KHSO4, water and brine. The organic layer was then dried with anhydrous Na2SO4 and evaporated under reduced pressure to obtain the crude product which was purified by column chromatography (eluent: DCM/MeOH (94:6), Rf: 0.6) to afford 16 as a yellow solid.

Yield: 0.62 g, 83%; [α]²⁷D: −153° (c=0.4, DMSO); mp: 215-217° C.; ¹H NMR (400 MHz, DMSO-d6) δ: 12.03 (br. s., 1H) 9.75 (br. s., 1H) 8.52 (br. s., 1H) 7.69-8.26 (m, 5H) 7.47 (br. s., 1H) 7.06-7.37 (m, 5H) 7.02 (br. s., 1H) 6.88 (br. s., 1H) 6.72 (br. s., 1H) 6.53 (br. s., 1H) 4.26 (m, 5H) 3.59 (s, 3H) 3.33-3.38 (q, 4H) 3.16 (m, 2H) 2.96 (m, 2H) 2.73 (m, 1H) 1.50 (m, 9H) 1.08-1.35 (m, 19H) 0.83 (m, 10H); ¹³C NMR (100 MHz, DMSO-d6) δ: 172.87, 172.30, 171.69, 171.04, 170.82, 159.41, 156.59, 156.32, 155.33, 154.89, 151.75, 143.53, 138.27, 130.79, 129.20, 128.02, 126.19, 109.74, 107.79, 103.90, 95.84, 78.18, 55.87, 51.86, 50.19, 47.87, 44.32, 39.93, 39.30, 31.56, 29.11, 28.11, 24.18, 22.76, 21.26, 19.23, 18.13, 17.94, 12.36; HRMS (ESI) calcd [M+H]+ for C53H75N10O12 1043.5560, found 1043.5548.

### Advantages of the Invention

The novel fluorophore conjugated ureidopyrimidinone-fluorescent and self-assembling amino acid of general formula (I) as potential building blocks for multiple application purposes.

The ureidopyrimidinone with self-complementary quadruple H-bonding is capable of undergoing efficient self-assembly.

Coumarin or hydroquinone conjugated ureidopyrimidinone-fluorescent amino acid can be incorporated into proteins and peptides.

Fluorescent amino acid would serve as a distinctive and useful amino acid building block for inducing peptide/protein assembly and recognition, and also to modulate protein/peptide secondary and tertiary structures which could be directly monitored by the handy tool fluorescence.

1. A fluorescent and self-assembling ureidopyrimidinone compound of formula (I) 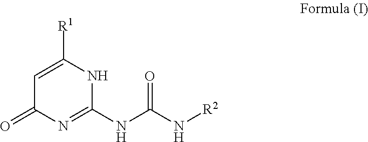
wherein:
R¹ represents 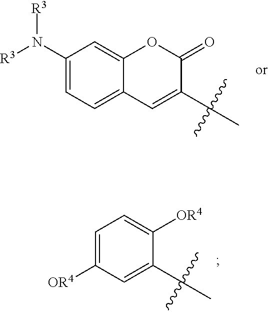
R² is selected from (un)substituted C₁-C₆ alkyl, (un)substituted aryl, (un)substituted natural amino acids which may be protected, (—CH₂)n-CH—(NH₂)(COOH), protected (—CH₂)n-CH—(NH₂)(COOH), linear or branched (un)substituted C₁-C₆ alcohols, or linear or branched (un)substituted C₁-C₆ amines; wherein n is 1-5; and
R³ and R⁴ are independently selected from (un)substituted C₁-C₆ alkyl, (un)substituted aryl, (un)substituted natural amino acids which may be protected, linear or branched (un)substituted C₁-C₆ alcohols, or linear or branched (un)substituted C₁-C₆ amines;
provided that at least one of R₂ or R₃ recite (—CH₂)n-CH—(NH₂)(COOH), protected (—CH₂)n-CH—(NH₂)(COOH), (un)substituted natural amino acids which may be protected, linear or branched (un)substituted C₁-C₆ alcohols, or linear or branched (un)substituted C₁-C₆ amines.

2. The compound as claimed in claim 1, wherein R¹ is 7-diethylamino-2-oxo-2H-chromen-3-yl or 1,4-dimethoxy phenyl.

3. The compound as claimed in claim 1, wherein R² is (un)substituted natural amino acids which may be protected and linear or branched (un)substituted C₁-C₆ amines.

4. The compound as claimed in claim 1, wherein the amino acid is selected from lysine, alanine, leucine, valine, phenylalanine, or combinations thereof.

5. The compound as claimed in claim 1, wherein the compound of formula (I) is selected from the group consisting of:
i. N6-((4-(7-(diethylamino)-2-oxo-2H-chromen-3-yl)-6-oxo-1,6-dihydropyrimidin-2-yl) carbamoyl) lysine (1);
ii. N6-((6-(2,5-dimethoxyphenyl)-4-oxo-1,4-dihydropyrimidin-2-yl)carbamoyl)lysine (2);
iii. N2-(tert-butoxycarbonyl)-N6-((4-(7-(diethylamino)-2-oxo-2H-chromen-3-yl)-6-oxo-1,6-dihydropyrimidin-2-yl)carbamoyl)lysine (6);
iv. N2-(tert-butoxycarbonyl)-N6-((6-(2,5-dimethoxyphenyl)-4-oxo-1,4-dihydropyrimidin-2-yl)carbamoyl)lysine (10);
v. Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylleucinate (11);
vi. Methyl N6-((4-(7-(diethylamino)-2-oxo-2H-chromen-3-yl)-6-oxo-1,6-dihydropyrimidin-2-yl)carbamoyl)lysylleucinate (12);
vii. Methyl N2-((tert-butoxycarbonyl)alanyl)-N6-((4-(7-(diethylamino)-2-oxo-2H-chromen-3-yl)-6-oxo-1,6-dihydropyrimidin-2-yl)carbamoyl)lysylleucinate (13);
viii. Methyl N2-(tert-butoxycarbonyl)-N6-((4-(7-(diethylamino)-2-oxo-2H-chromen-3-yl)-6-oxo-1,6-dihydropyrimidin-2-yl)carbamoyl)lysylalanylleucinate (14);
ix. Methyl N6-((4-(7-(diethylamino)-2-oxo-2H-chromen-3-yl)-6-oxo-1,6-dihydropyrimidin-2-yl)carbamoyl)lysylalanylleucinate (15); and
x. Methyl N2-(tert-butoxycarbonyl)phenylalanylvalyl-N6-((4-(7-(diethylamino)-2-oxo-2H-chromen-3-yl)-6-oxo-1,6-dihydropyrimidin-2-yl)carbamoyl)lysylalanylleucinate (16).

6. A process for synthesis of fluorescent and self-assembling ureidopyrimidinone compound, the process comprising the steps of:
a) reacting compound of formula 4′ with carbonyldiimidazole (CDI) in a solvent to obtain the intermediate of formula 5′; 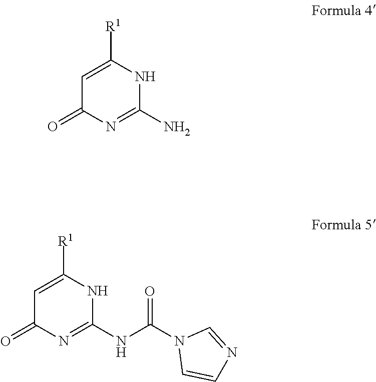
b) reacting the intermediate of formula 5′ as obtained in step (a) with Na-Boc-protected amino acid (Na-Boc-L-lysine) in presence of base in solvent to obtain BOC-protected compound of formula 6′; 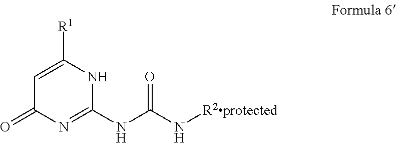
c) deprotecting the compound of formula 6′ as obtained in step (a) by using equal proportion of TFA and DCM to obtain the compound of formula (I); 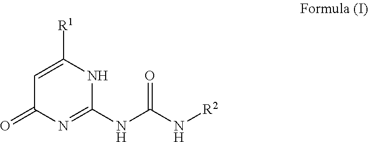
wherein:
R¹ represents 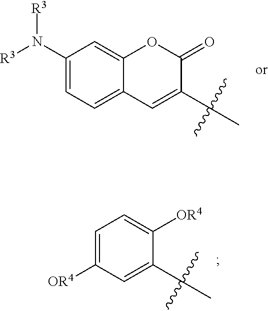
R² is selected from (un)substituted C₁-C₆ alkyl, (un)substituted aryl, (un)substituted natural amino acids which may be protected, (—CH₂)n-CH—(NH₂)(COOH), protected (—CH₂)n-CH—(NH₂)(COOH), linear or branched (un)substituted C₁-C₆ alcohols, or linear or branched (un)substituted C₁-C₆ amines; wherein n is 1-5; and
R³ and R⁴ are independently selected from (un)substituted C₁-C₆ alkyl, (un)substituted aryl, (un)substituted natural amino acids which may be protected, linear or branched (un)substituted C₁-C₆ alcohols, or linear or branched (un)substituted C₁-C₆ amines;
provided that at least one of R₂ or R₃ recite (—CH₂)n-CH—(NH₂)(COOH), protected (—CH₂)n-CH—(NH₂)(COOH), (un)substituted natural amino acids which may be protected, linear or branched (un)substituted C₁-C₆ alcohols, or linear or branched (un)substituted C₁-C₆ amines.

7. The process as claimed in claim 6, wherein the solvent is selected from polar or non-polar, or protic or aprotic solvents.

8. The process as claimed in claim 6, wherein the solvent is selected from dimethyl sulfoxide, lower alcohols, nitriles, ketones, halogenated hydrocarbons, trifluoro acetic acid(TFA), or combinations thereof.

9. The process as claimed in claim 6, wherein the base is selected from ethylamine, triethylamine, N,N-diisopropylethylamine(DIPEA), pyridine, sodium hydroxide, alkali or alkaline earth metal carbonates, alkali or alkaline earth metal bicarbonates, or combinations thereof.

10. The process as claimed in claim 6, wherein the process further comprises a step of incorporating the compound of formula (I) into peptides by reacting the compound of formula (I) with amino acids using a coupling reagent, wherein the coupling reagent is hexafluorophosphate benzotriazoletetramethyluronium (HBTU).
